(19) United States
(12) Patent Application Publication
(10) Pub. No.: US 2024/0415684 A1
(43) Pub. Date: Dec. 19, 2024

(54) ADJUSTABLE JOINT BRACE WITH TELESCOPICALLY ADJUSTABLE LENGTH

(71) Applicants: Hongshi WU, Shenzhen (CN); Botao CHENG, Shenzhen (CN); Zhipeng ZHU, Shenzhen (CN)

(72) Inventors: Hongshi WU, Shenzhen (CN); Botao CHENG, Shenzhen (CN); Zhipeng ZHU, Shenzhen (CN)

(21) Appl. No.: 18/633,531

(22) Filed: Apr. 12, 2024

(30) Foreign Application Priority Data
Jun. 19, 2023 (CN) 2023215648257

(51) Int. Cl. A61F 5/01

(52) U.S. Cl. CPC A61F 5/0102; A61F 2005/0137; A61F 2005/0179

(57) ABSTRACT

An adjustable joint brace with a telescopically adjustable length, having a strap and a plurality of supports; the strap has first and second straps connected through a rotatable adjustment device so that an angle between the first and second straps is adjustable; buckling holes spaced with one another are arranged on the strap along a lengthwise direction; each support has an elastic buckle buckled to one of the buckling holes selected as desired, so each support is fastened onto the strap. The joint brace is adaptable to patients with different heights and sizes and adaptable to different joints.

### BACKGROUND OF THE INVENTION

The present utility model relates to a joint brace with an adjustable length.

At present, joint braces are usually worn for nursing joint injuries and fixing the injured joints. Since different people have different joint sizes and different joints that need to be braced, a joint brace adaptable to different people is required.

### BRIEF SUMMARY OF THE INVENTION

In order to overcome the above technical problems, the present utility model provides a joint brace with an adjustable length. It is achieved by the following technical solutions.

An adjustable joint brace with a telescopically adjustable length, comprising a strap and a plurality of supports, wherein the strap comprises a first strap and a second strap; the first strap is connected to the second strap through a rotatable adjustment device; a plurality of buckling holes spaced with one another are arranged on the strap along a lengthwise direction of the strap; each of the supports is provided with an elastic buckle, and the elastic buckle of each of the supports is selectively buckled and fastened to one of the buckling holes, so each of the supports is fastened onto the strap.

Preferably, the joint brace also comprises a plurality of bandages corresponding to the supports respectively; in each of the supports, one end of the support is connected to one end of a corresponding bandage, another end of the corresponding bandage is connected with a fastener, and said another end of the corresponding bandage is connected to another end of the support through the fastener; each fastener is provided with a buckling case, and a protruding rod is provided on a corresponding support; a buckling slot is arranged at one side of the protruding rod; the buckling case is provided with a slidable clamping block; a biasing spring is arranged between the slidable clamping block and the buckling case; the buckling case is fitted on the protruding rod, and the slidable clamping block slides into the buckling slot due to biasing force of the biasing spring, and the biasing spring keeps the slidable clamping block in the buckling slot.

The present utility model has the beneficial effects below: According to the present utility model, the first strap and the second strap are connected through the rotatable adjustment device, so that an angle between the first strap and the second strap can be adjusted by rotating the rotatable adjustment device. In the present utility model, the strap is provided with the plurality of buckling holes, and each of the supports can be selectively buckled to a buckling hole as desired by buckling the elastic buckle to a selected buckling hole, and further, by buckling the supports specifically at two ends of the strap to different buckling holes, a length of the joint brace can be telescopically adjusted; accordingly, the joint brace is adaptable to patients with different heights and sizes and also adaptable to different joints.

### BRIEF DESCRIPTION OF THE DRAWINGS

FIG. 1 is a structural view of a joint brace according to the present utility model;

FIG. 2 is a partial structural view of a joint brace according to the present utility model, where a connecting position between a fastener and a corresponding support is sectioned to show a cross sectional structure, and where bandages are omitted from the figure.

### DETAILED DESCRIPTION OF THE INVENTION

The present utility model is further illustrated with reference to the following embodiments which only illustrate some embodiments of the present utility model, and these embodiments are only for explaining the present utility model and do not limit the scope of the present utility model.

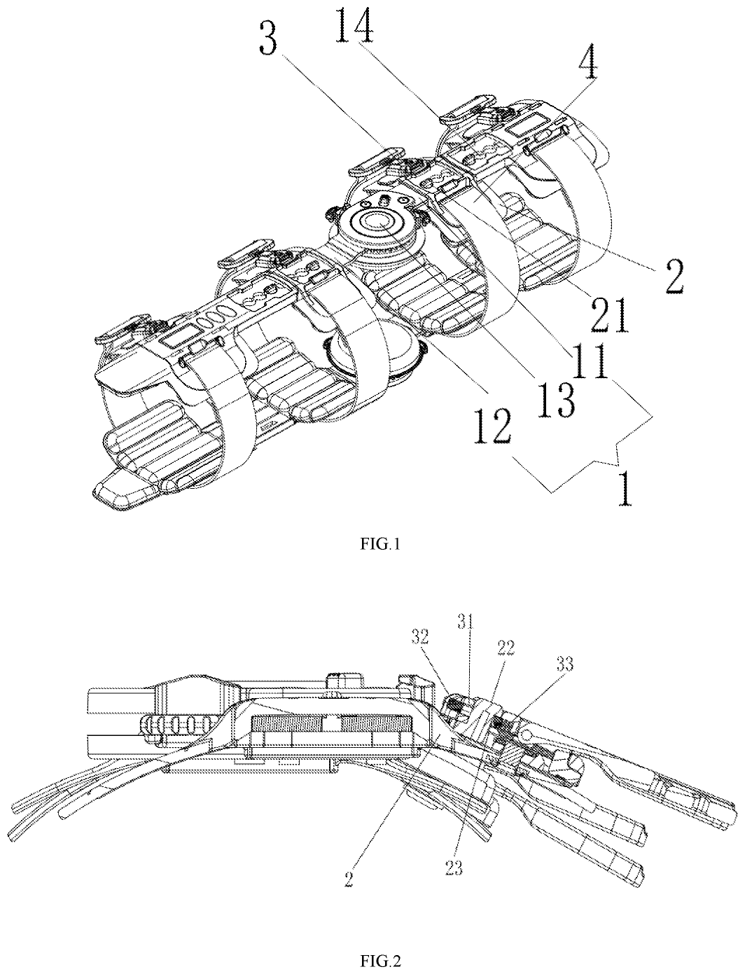

As shown in FIG. 1 to FIG. 2, an adjustable joint brace with a telescopically adjustable length comprises a strap 1 and a plurality of supports 2, wherein the strap 1 comprises a first strap 11 and a second strap 12; the first strap 11 is connected to the second strap 12 through a rotatable adjustment device 13; a plurality of buckling holes 14 spaced with one another are arranged on the strap 1 along a lengthwise direction of the strap 1; each of the supports 2 is provided with an elastic buckle 21, and the elastic buckle 21 of each of the supports 2 is selectively buckled and fastened to one of the buckling holes 14, so each of the supports 2 is fastened onto the strap 1.

According to the present utility model, the first strap 11 and the second strap 12 are connected through the rotatable adjustment device 13, so that an angle between the first strap 11 and the second strap 12 can be adjusted by rotating the rotatable adjustment device 13; besides, in the present utility model, the strap 1 is provided with the plurality of buckling holes 14, and each of the supports 2 can be selectively buckled to a buckling hole 14 as desired by buckling the elastic buckle 21 to a selected buckling hole 14, and further, by buckling the supports 2 specifically at two ends of the strap 1 to different buckling holes 14, a length of the joint brace can be telescopically adjusted; accordingly, the joint brace is adaptable to patients with different heights and sizes and also adaptable to different joints.

According to one embodiment, the joint brace also comprises a plurality of bandages 4 corresponding to the supports 2 respectively; in each of the supports 2, one end of the support 2 is connected to one end of a corresponding bandage 4, another end of the corresponding bandage 4 is connected with a fastener 3, and said another end of the corresponding bandage 4 is connected to another end of the support 2 through the fastener 3, so that each support 2 together with the corresponding bandage 4 and said fastener 3 formed on the corresponding bandage 4 form a loop configured to be fixed on a human body part; each fastener 3 is provided with a buckling case 31; a protruding rod 22 is provided on a corresponding support 2; a buckling slot 23 is arranged at one side of the protruding rod 22; the buckling case 31 is provided with a slidable clamping block 32; a biasing spring 33 is arranged between the slidable clamping block 32 and the buckling case 31; when the buckling case 31 is fitted on the protruding rod 22; the slidable clamping block 32 slides into the buckling slot 23 due to biasing force of the biasing spring 33; and the biasing spring 33 keeps the slidable clamping block 32 in the buckling slot 23. During actual use, the strap 1 is placed over a joint where fixation is required, and then said another end of each bandage 4 connected with the fastener 3 is connected to said another end of a corresponding support 2 through the fastener 3, so that the bandage 4 together with fastener 3 and the corresponding support 2 form a loop configured to be fixed on a human body part. In the present utility model, the fastener 3 can be quickly installed and fixed to the corresponding support 2, and the operation is more convenient, fast, and reliable.

The above description is merely the preferred embodiments of the present utility model and is not intended to make any form of limitation on the present utility model. Although the present utility model has been disclosed with the preferred embodiments as mentioned above, it is not intended to limit the present utility model. Any of those skilled in the art, without departing from the technical scope of the present utility model, may make some changes or modifications according to the disclosed technical content to form equivalent embodiments. As long as these simple amendments, equivalent changes, and modifications fall within the contents of the technical solutions of the present utility model according to the technical substance of the present utility model, they shall all fall within the scope of the technical solutions of the present utility model.

What is claimed is:

1. A joint brace, which is adjustable and with a telescopically adjustable length;
wherein the joint brace comprises a strap and a plurality of supports; the strap comprises a first strap and a second strap; the first strap is connected to the second strap through a rotatable adjustment device; a plurality of buckling holes spaced with one another are arranged on the strap along a lengthwise direction of the strap; each of the supports is provided with an elastic buckle, and the elastic buckle of each of the supports is selectively buckled and fastened to one of the buckling holes, so each of the supports is fastened onto the strap.

2. The joint brace of claim 1, also comprising a plurality of bandages corresponding to the supports respectively; in each of the supports, one end of the support is connected to one end of a corresponding bandage, another end of the corresponding bandage is connected with a fastener, and said another end of the corresponding bandage is connected to another end of the support through the fastener; each fastener is provided with a buckling case, and a protruding rod is provided on a corresponding support; a buckling slot is arranged at one side of the protruding rod; the buckling case is provided with a slidable clamping block; a biasing spring is arranged between the slidable clamping block and the buckling case; the buckling case is fitted on the protruding rod, and the slidable clamping block slides into the buckling slot due to biasing force of the biasing spring, and the biasing spring keeps the slidable clamping block in the buckling slot.
